(19) United States
(12) Patent Application Publication
(10) Pub. No.: US 2007/0134666 A1
(43) Pub. Date: Jun. 14, 2007

(54) Simultneous analytical method for oxidatively damaged guanine compound and concentration correcting substance thereof, and analyzer used for this analytical method

(76) Inventor: Hiroshi Kasai, Fukuoka-ken (JP)

Correspondence Address: DAY PITNEY LLP, 7 TIMES SQUARE, NEW YORK, NY, 10036-7311, US

(21) Appl. No.: 10/576,967

(22) PCT Filed: Oct. 26, 2004
(86) PCT No.: PCT/JP04/15826

§ 371 Date: Aug. 21, 2006

(30) Foreign Application Priority Data
Oct. 27, 2003 (JP) 2003-366220
Apr. 30, 2004 (JP) 2004-135791
Aug. 2, 2004 (JP) 2004-225661

(51) Int. Cl. C12Q 1/68; C12M 3/00

(52) U.S. Cl. 435/6; 435/287.2

(57) ABSTRACT

An object of the present invention is to provide a method of efficiently analyzing an oxidatively damaged guanine compound and a concentration correcting substance for this oxidatively damaged guanine compound, and an analyzer for implementing this method. The present invention provides an analytical method characterized by a step to purify an oxidatively damaged guanine compound generated as a result of damaging guanine in DNA, RNA or nucleotide contained in a sample; a step to measure a concentration correcting substance for the oxidatively damaged guanine compound contained in the sample by a UV detector; a step to measure the oxidatively damaged guanine compound by a detector; and by simultaneously analyzing the oxidatively damaged guanine compound and the concentration correcting substance for the oxidatively damaged guanine compound.

### BACKGROUND OF THE INVENTION

1. Field of the Invention

The present invention relates to a simultaneous analytical method for an oxidatively damaged guanine compound and a concentration correcting substance thereof, particularly a simultaneous analytical method for 8-hydroxydeoxyguanosines (hereunder referred to as “8-OH-dG”) and 7-methylguanine (hereunder referred to as “7-MG”) or creatinine (hereafter, referred to as “Cre”), and an analyzer for performing such.

Priority is claimed on Japanese Patent Application No. 2003-366220, filed Oct. 27, 2003, No. 2004-135791, filed Apr. 30, 2004, and No. 2004-225661, filed Aug. 2, 2004, the content of which is incorporated herein by reference.

2. Description of Related Art

Recently, the effects of active oxygen in vivo have been well researched. Normally, active oxygen acts as a defense system when a foreign body invades a living organism. However, if excessive active oxygen is generated due to food additives (carcinogen), air pollution, smoking, stress, and the like, it causes DNA damage and produces 8-OH-dG, which is a type of oxidative by DNA damaged product. This 8-OH-dG is considered to induce mutation and plays an important role in the carcinogenesis process. Moreover, attention has been paid to the active oxygen as a causative factor not only of carcinogenesis but also of various disorders or aging.

Therefore, by knowing the amount of active oxygen in vivo, individual carcinogenesis risk evaluation, prediction and diagnosis of various disorders relating to the active oxygen, evaluation of the degree of aging or general health can be performed.

However, since the active oxygen in vivo is unstable, it is difficult to directly detect. Therefore, as an index of the active oxygen, it has been proposed to measure 8-OH-dG produced by the active oxygen. Analysis methods for 8-OH-dG reported so far can be largely classified into six types, including; (1) a method of analyzing a fraction purified by an affinity column having anti-8-OH-dG antibodies, using HPLC-ECD, (2) a method of finally detecting 8-OH-dG using HPLC-ECD with a column switching method where a carbon column is connected between two reversed phase columns (Non-patent document 1), and (3) a method of detecting [8-OH-dG) using ECD by connecting a multifunction column (a gel filtration column having both functions of a reverse phase column and a cation-exchange column) and a reverse phase column through a sampling injector (an apparatus that collects a specific fraction and injects a specific amount into columns after mixing) (Non-patent document 2).

[Non-patent document 1] Bogdanov, M. B., et al., Free Rad. Biol. Med., 1999, Vol. 27, p. 647-666

[Non-patent document 2] Kasai, H., et al., Jpn. J. Cancer Res., 2001, Vol. 92, p. 9-15

[Non-patent document 3] Topp, H., et al., Anal. Biochem., 1987, Vol. 161, p. 49-56

### SUMMARY OF THE INVENTION

The value of 8-OH-dG in vivo temporally varies in many cases. When 8-OH-dG is accurately quantified, it is necessary to prepare a sample for 24 hours or to correct the 8-OH-dG concentration using a concentration correcting substance for 8-OH-dG.

In the above-methods, when 8-OH-dG is quantified by correcting the concentration using a concentration correcting substance, the sample is placed into at least two containers, one for analyzing the 8-OH-dG itself and the other for analyzing a concentration correcting substance (for example, creatinine (Cre)). The value calculated from the measured values of both substances (for example, (8-OH-dG/Cre) is regarded as the index of oxidative DNA damage, and complex procedures are required for analysis of the collection, storage and the analysis of samples and data analysis.

The present invention has been accomplished by taking the related circumstances into consideration, and has the object of providing an analytical method for efficiently analyzing oxidatively damaged guanine compound and a concentration correcting substance for the oxidatively damaged guanine compound, and an analyzer for performing the same.

The present inventor discovered that above-mentioned problem could be resolved using a method of simultaneously analyzing 8-OH-dG and 7-methylguanine or creatinine, which is a concentration correcting substance for 8-OH-dG, and an apparatus used for this method, as a result of keen examination, by taking the circumstances into consideration, and completed the present invention.

The substance, 7-MG, is an RNA decomposition product relating to the metabolic rate (MR), known to be excreted into the urine at a specific rate along with pseudouridine, which is an RNA catabolite (Non-patent Literature 3). It is also known that there is a strong correlation between the creatinine concentration and the 7-MG concentration. Therefore, (8-OH-dG/7-MG) where the 8-OH-dG value is corrected by 7-MG, becomes an index of oxidative DNA damage having the same level of reliability as the value (8-OH-dG/Cre), where the 8-OH-dG value is corrected by creatinine.

Similarly, it is considered that [the 8-OH-dG value] is also correctable with other catabolite, such as pseudouridine, N2, N2-dimethylguanosine, or N6-threoninocarbonyladenosine.

In other words, the first invention of the present invention is an analytical method for an oxidatively damaged guanine compound characterized by a step to purify an oxidatively damaged guanine compound generated as a result of damaging guanine in DNA, RNA or nucleotide contained in a sample using an anion-exchange column (HPLC-1) and a step to measure the oxidatively damaged guanine compound with a detector.

Since this composition enables simple analysis of the oxidatively damaged guanine compound, it becomes possible to reduce the required labor and time by half in the collection, storage, analysis of samples and data analysis.

Further, the present invention does not require a large quantity of eluent or a washing solution, and the generation of a toxic waste fluid is also small, superior from an environmental aspect.

The second invention of the present invention is the analytical method for an oxidatively damaged guanine compound according to the first invention where the oxidatively damaged guanine compound is 8-hydroxydeoxyguanosine (8-OH-dG) and/or 8-hydroxyguanine (8-OH-Gua).

The third invention of the present invention is an analytical method for an oxidatively damaged guanine compound characterized by a step to purify an oxidatively damaged guanine compound generated as a result of damaging guanine in DNA, RNA or nucleotide contained in a sample using an anion exchange column (HPLC-1); a step to measure a concentration correcting substance for the oxidatively damaged guanine compound contained in the sample by a detector; and a step to measure the oxidatively damaged guanine compound; and by simultaneously analyzing the oxidatively damaged guanine compound and the concentration correcting substance.

With this method, since the oxidatively damaged guanine compound and the concentration correcting substance for the oxidatively damaged guanine compound can be simultaneously analyzed, it becomes possible to reduce the required labor and time by half in the collection, storage and analysis of samples and data analysis.

The fourth invention of the present invention is the analytical method for an oxidatively damaged guanine compound according to the third invention where the oxidatively damaged guanine compound is 8-hydroxydeoxyguanosine (8-OH-dG) and/or 8-hydroxyguanine (8-OH-Gua), and the concentration correcting substance for the oxidatively damaged guanine compound is 7-methylguanine (7-MG) and/or creatine (Cre).

The fifth invention of the present invention is an analytical method for an oxidatively damaged guanine compound characterized by a step to purify an oxidatively damaged guanine compound generated as a result of damaging guanine in DNA, RNA or nucleotide contained in a sample using an anion exchange column (HPLC-1); a step to detect an elution position of a marker pre-added into the sample and to appropriately measure a concentration correcting substance for the oxidatively damaged guanine compound contained in the sample by a detector; and a step to measure the oxidatively damaged guanine compound by [another] detector; and simultaneously analyzing the oxidatively damaged guanine compound and the concentration correcting substance for the oxidatively damaged guanine compound.

With this method, since a substance, such as 8-hydroxyguanosine (ribonucleoside) (8-OH-rGuo) is pre-added to a sample as an internal standard marker of 8-OH-dG, it becomes possible to more accurately regulate the elution position (time) from a column and the fractionation range of an eluent for an oxidatively damaged guanine compound. As a result, 8-OH-dG can be measured with high accuracy and high reproducibility.

The sixth invention of the present invention is the analytical method for an oxidatively damaged guanine compound according to the fifth invention where the oxidatively damaged guanine compound is 8-hydroxydeoxyguanosine (8-OH-dG) and/or 8-hydroxyguanine (8-OH-Gua), and the concentration correcting substance for the oxidatively damaged guanine compound is 7-methylguanine (7-MG) and/or creatine in (Cre), and the marker is 8-hydroxyguanosine (ribonucleoside) (8-OH-rGuo).

The seventh, eighth and ninth inventions of the present invention are the analytical methods according to the first, third and fifth inventions respectively characterized by the fact that the sample is urine.

The tenth, eleventh and twelfth inventions of the present invention are the analytical methods for an oxidatively damaged guanine compound according to the seventh, eighth and ninth inventions where analytical is conducted by re-extracting the urine onto a piece of paper and dried, enabling collection of samples at any location, making it possible to efficiently collect a large quantity of samples in an analytical laboratory via postal mail.

The thirteenth invention of the present invention is an analytical method for an oxidatively damaged guanine compound according to any one of the first to twelfth inventions where a carboxylic acid type column and an eluent containing carboxylic acid or salt thereof are used in the step to accomplish purification using the anion exchange column (HPLC-1). With this method, the elution positions of 8-OH-dG and 8-OH-Gua in the HPLC-1 can overlap, making it possible to simultaneously analyze both substances.

The fourteenth invention of the present invention is the analytical method according to any one of the first to twelfth inventions additionally comprising a step to further purify a fraction containing the oxidatively damaged guanine compound, purified using the anion exchange column (HPLC-1), using a reverse phase column (HPLC-2); and a step to measure the purified oxidatively damaged guanine compound purified using the HPLC-2.

In this case, measurement of the oxidatively damaged guanine compound (such as 8-hydroxydeoxyguanosines (8-OH-dG)) purified using the HPLC-1 is carried out in the order of; (1) the peak recognition of ribonucleosides 8-OH-rGuo in anion-exchange chromatography, (2) starting of 8-OH-dG fractionation after a fixed time, (3) completion of 8-OH-dG fractionation after a fixed time, and (4) optionally mixing 8-OH-dG fraction, and then injected into a reverse phase column.

The analyzer of the present invention does not require any major mechanism, is inexpensive and also excels in economical efficiency.

The fifteenth invention of the present invention is an analyzer for an oxidatively damaged guanine compound characterized by composing 1) an anion-exchange column (HPLC-1) that specifically absorbs an oxidatively damaged guanine compound generated as a result of damaging guanine in DNA, RNA or nucleotide contained in a sample, 2) a reverse phase column (HPLC-2) that further purifies the fraction containing the oxidatively damaged guanine compound obtained by purification using the anion-exchange column (HPLC-1), and 3) a detector used for obtaining a fraction containing the oxidatively damaged guanine compound [eluted] from the anion-exchange column (HPLC-1) and another detector that measures the purified oxidatively damaged guanine compound obtained from the reverse phase column (HPLC-2).

The sixteenth invention of the present invention is an analyzer according to the fifteenth invention where the detector used for obtaining the fraction containing the oxidatively damaged guanine compound from the anion-exchange column (HPLC-1) comprises a detector equipped with a cell having a short optical path.

The seventeenth invention of the present invention is an analytical mechanism (including a control program) for an oxidatively damaged guanine compound for receiving the peak signal of a marker pre-added to a sample from a detector;

transmitting a signal to open a valve when the oxidatively damaged guanine compound is eluted after a fixed time;

starting fractionation;

further transmitting a fractionation completion signal after another fixed time;

then, transmitting a signal to inject the obtained fraction containing the oxidatively damaged guanine compound into a reverse phase column (HPLC-2); and

purifying and recovering the oxidatively damaged guanine compound eluted from the reverse-phase column (HPLC-2).

This analytical mechanism (including a control program) enables automation of the efficient and accurate analysis of an oxidatively damaged guanine compound by utilizing in the analyzer of the present invention.

In a method of analyzing an oxidatively damaged guanine compound generated in the process of damaging DNA, RNA or nucleotide, if the analytical method of the present invention is used, the oxidatively damaged guanine compound generated as a result of damaging guanine in DNA, RNA or nucleotide and a concentration correcting substance for the oxidatively damaged guanine compound can be (simultaneously) analyzed, reducing the labor and time by half in the collection, storage and analysis of samples and data analysis, making it possible to more efficiently analyze the oxidatively damaged guanine compound.

Moreover, with the analytical method of the present invention, accuracy is high, reproducibility is excellent, and analytical time is also short, and continuous operation enables mass-processing.

The analyzer of the present invention can provide an analytical method that proves the above effects, is low in cost with superior economical efficiency.

In the present application, the “oxidatively damaged guanine compound” includes both an “oxidatively damaged guanine nucleotide” and “oxidatively damaged guanine”.

### BRIEF DESCRIPTION OF THE DRAWINGS

FIG. 1 is a schematic diagram showing an example of an apparatus for simultaneously analyzing 8-OH-dG (8-OH-Gua) and 7-MG or Cre.

FIG. 2 is a schematic diagram showing another example of an apparatus for simultaneously analyzing 8-OH-dG (8-OH-Gua) and 7-MG or Cre.

FIG. 3 is a chart showing an example of 8-OH-dG fractionation based upon a peak of 8-OH-rGuo, which is a marker, and measurement of 7-MG or Cre (HPLC-1).

FIG. 4 is a chart showing a measurement example of 8-OH-dG relating to the implementation of the present invention (HPLC-2).

FIG. 5 is a chart showing an example of simultaneously analyzing Cre, 7-MG, 8-OH-Gua and 8-OH-dG

In the above figures, numeric symbol 11 indicates the anion-exchange column (HPLC-1), 12 indicates the reverse phase column (HPLC-2), 13 indicates the electrochemical detector, 14 indicates the UV detector, 15 indicates the switching valve, 16 indicates the switching valve, 17 indicates the automatic sampler, 27 indicates the sampling injector, respectively.

### BEST MODE FOR CARRYING OUT THE INVENTION

(Oxidatively Damaged Compound in DNA, RNA or Nucleotide)

An oxidatively damaged compound in DNA, RNA or nucleotide, including 8-OH-dG, is generated as a result of damaging DNA, RNA or nucleotide by an active oxygen (oxygen radical) and the like in vivo, and is used as the index of active oxygen. Oxidatively damaged compounds other than 8-OH-dG include 2-hydroxydeoxyadenosine (2-OH-dA) include 5-hydroxydeoxycytidine (5-OH-dC), 5-formyldeoxyuridine (5-CHO-dU), 8-OH-rGuo, 8-hydroxyguanine (8-OH-Gua) and the like, excreted from the organism as an undesired substance via the urine. Among these, oxidatively damaged guanine compounds, such as 8-OH-dG, 8-OH-rGuo or 8-OH-Gua, are negatively charged, to be easily purified and recovered by an anion-exchange column described in the following paragraph. Among these, it is preferable to use 8-OH-dG as the active oxygen index. The oxidatively damaged guanine compound in the present application is generated as a result of damaging guanine in DNA, RNA or nucleotide by active oxygen, such as 8-OH-dG, 8-OH-rGuo or 8-OH-Gua, and the oxidatively damage means hydroxylation.

(Samples)

Samples used for the analytical method of the present invention to (simultaneously) analyze an oxidatively damaged guanine compound (including 8-OH-dG) and appropriately a concentration correcting substance for the oxidatively damaged guanine compound may include all biological samples, such as urine, serum, cerebrospinal fluid, saliva, or a medium after culturing the cells. Among these, urine is preferable because it is easy to collect and the oxidatively damaged guanine compound is stable therein.

The analytical method and the analyzer for performing it according to the present invention are described next. The analytical method of the present invention comprises a step to purify an oxidatively damaged guanine compound (such as, 8-OH-dG); a step to appropriately measure a concentration correcting substance for the oxidatively damaged guanine compound (such as, 7-MG or Cre); and a step to measure the oxidatively damaged guanine compound. Moreover, the analyzer of the present invention is composed of members for the above purification and measurement.

Hereafter, 8-OH-dG as the oxidatively damaged guanine compound and 7-MG and Cre as the concentration correcting substance for the oxidatively damaged guanine compound are described as examples.

(Analyzer)

An apparatus relating to an embodiment of the present invention to analyze 8-OH-dG, and appropriately 7-MG and/or Cre is equipped with:
1) an anion-exchange column that specifically absorbs 8-OH-dG (HPLC-1),
2) a detector, such as UV detector, that detects a marker, such as of 8-OH-rGuo, which is an index of the elution position of 8-OH-dG, and that measures 7-MG or Cre,
3) a reverse phase column that further purifies a fraction containing 8-OH-dG obtained from the anion-exchange column (HPLC-1), and
4) a detector that measures the purified 8-OH-dG obtained from the reverse phase column (HPLC-2).

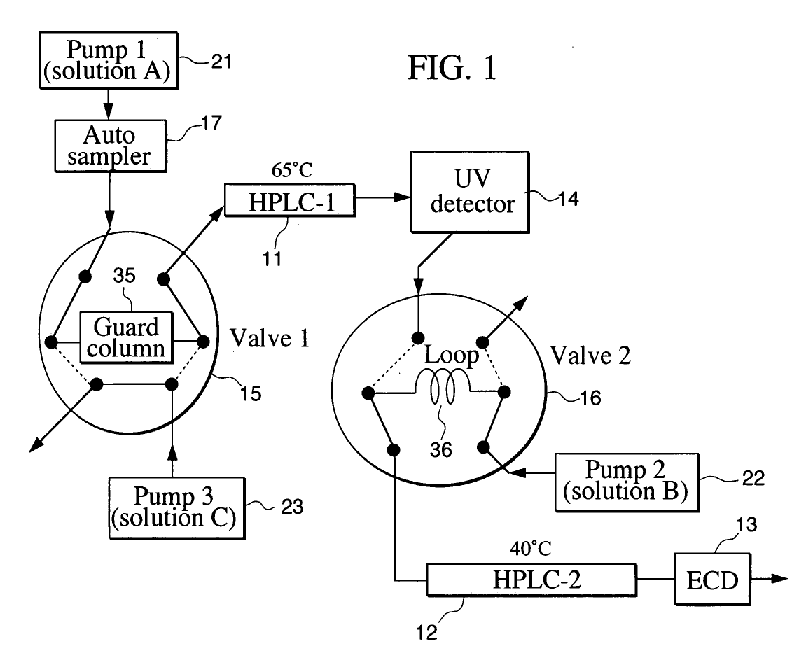

FIG. 1 is a schematic diagram showing an example of the analyzer of the present invention. In the diagram, reference symbol 11 is an anion-exchange column (HPLC-1), connected to a reverse phase column (HPLC-2) 12 via a UV detector 14 and a column switching valve 16. Moreover, upstream of the anion-exchange column (HPLC-1) 11 is connected a column switching valve 15 to which is connected an automatic sampler 17 for injecting samples.

Moreover, pumps 21, 22 and 23 are provided for sending eluents for eluting molecules absorbed onto the column (the eluent to be used for the anion-exchange column (HPLC-1) 11 is regarded as solution A, and the eluent to be used for the reverse phase column (HPLC-2) 12 is regarded as solution B) and a washing solution (solution C) for washing a guard column (filled with an anion-exchange resin, which is the same as that used in the anion-exchange column (HPLC-1) 11) connected to the column switching valve 15. The pump 21 is connected to the automatic sampler 17, the pump 22 is connected to the column switching valve 16, and the pump 23 is connected to the column switching valve 15.

In this example, instead of the automatic sampler 17, a sampling injector (“231XL” manufactured by Gilson) having a function to automatically operate the column switching valve 16 by peak detection of 8-OH-rGuo, can be used.

When implementing the method of the present invention, when two columns, HPLC-1 and HPLC-2, are used according to the column switching method, it is necessary to pre-inject a standard solution of an oxidatively damaged guanine compound (such as, 8-OH-dG) into the HPLC-1; to determine the starting and completion time of fractionation; and in urine analysis the oxidatively damaged guanine compound purified by the HPLC-1 under the same condition is injected into the HPLC-2.

In the meantime, when implementing this method, a new program can be loaded into the 231XL and measurements taken instead of the above method.

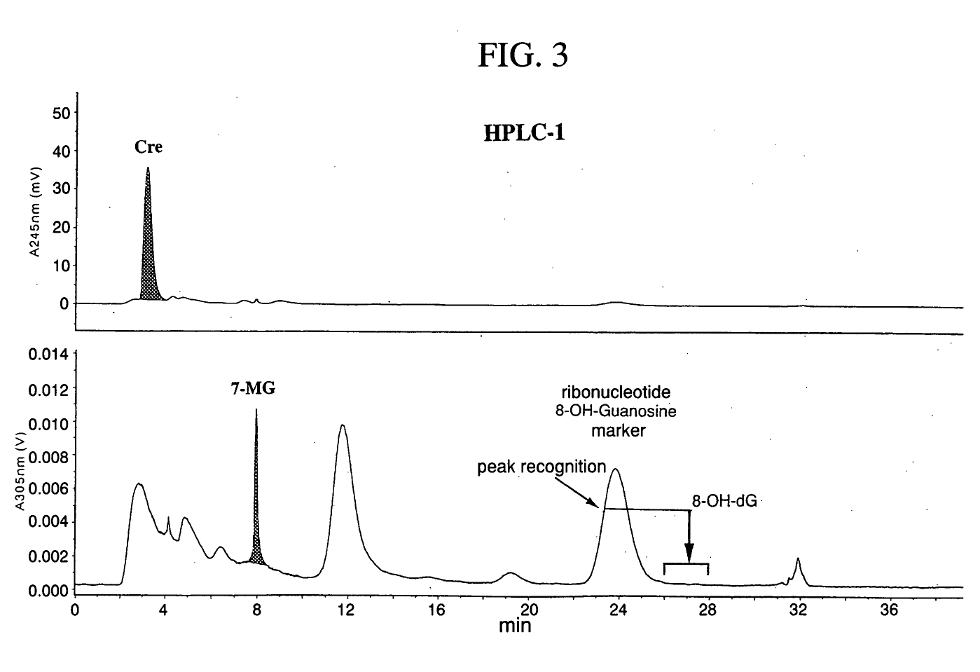

This program performs:
(1) peak recognition of a marker, such as ribonucleosides 8-OH-rGuo,
(2) starting of 8-OH-dG fractionation after a fixed time,
(3) completion of 8-OH-dG fractionation after a fixed time, and
(4) injection into the HPLC-2 (refer to FIG. 3), and it is unnecessary to pre-set the fractionation range (time) of the oxidatively damaged guanine compound, such as 8-OH-dG

Specifically, these functions are realized by the following flow.
(1) A sample is injected into the HPLC-1 by the 231XL.
(2) The system is kept standing-by for a preset time (T1).
(3) The 231XL starts monitoring the signal from the UV detector.
(4) The system is kept standing-by until exceeding the preset UV level (peak detection).
(5) After the peak detection, the system is kept standing-by for a preset time (T2). Then a contact signal is transmitted to a valve so as to start fractionation inside the loop.
(6) After a preset time (T3), the contact signal is transmitted to the valve. Then, fractionation is completed and at the same time the fraction inside the loop is injected into HPLC-2.

As described hereafter, due to using the sampling injector (“231XL” manufactured by Gilson), the fractionation range (time) of 8-OH-dG is automatically determined based on the relative position relative to 8-OH-rGuo, and it is not necessary to preset the fractionation range (time) of 8-OH-dG.

Since abovementioned anion-exchange column (HPLC-1) 11 specifically absorbs 8-OH-dG contained in the sample, the recovery rate is very high and almost all impurities can be removed, enabling fractions with few impurities to be obtained. According to the anion-exchange column (HPLC-1) 11, a negatively charged oxidatively damaged guanine compound, such as 8-OH-rGuo, 8-OH-Gua, etc., can also be easily purified and recovered. The anion-exchange column (HPLC-1) 11 is not specifically limited as long as an anion-exchange resin is used as the filler. Examples of the specific filler include styrenedivinylbenzene polymer combined with quaternary ammonium group, polyhydroxymethacrylate polymer combined with quaternary ammonium group, and the like. Moreover, examples of commercial filler include Aminex HPX-72S (manufactured by Bio-Rad), Shodex column filler (manufactured by Showa Denko K.K.), MCI GEL CA08F (manufactured by Mitsubishi Chemical Industries Ltd., Hamilton RCX-10), and the like.

As the particle diameter of the anion-exchange resin, even though excellent results can be obtained with a 7 μm particle diameter, using an anion-exchange resin with a smaller particle diameter (3 to 5 μm) results in higher separability, and also enables shortening of the column length and reduction of the analytical time.

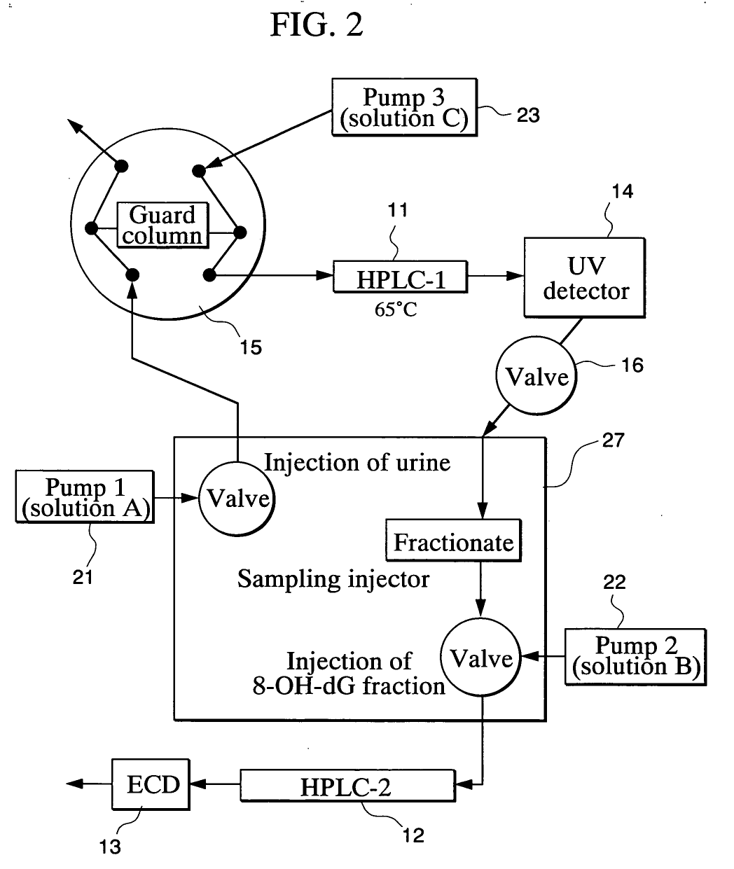

The internal diameter of the column that fills the anion-exchange resin is not specifically limited. This is preferably from approximately 1 mm to 1.5 mm. If the internal diameter of the column is from 2.0 to 4.6 mm, as shown in FIG. 2, it is preferable to use a sampling injector 27 (“233XL” manufactured by Gilson, or the like) connected to the column switching valve 16 so that the fraction containing 8-OH-dG is automatically injected into the reverse phase column (HPLC-2) 12 after the peak recognition of a marker, such as 8-OH-Guo. In order to perform this method, a new program was loaded into the 233XL and measurements taken. This program performs
(1) the peak recognition of marker, such as ribonucleosides 8-OH-rGuo,
(2) starting of 8-OH-dG fractionation after a fixed time,
(3) completion of 8-OH-dG fractionation after a fixed time,
(4) mixing of 8-OH-dG fraction, and
(5) injection into the HPLC-2.

Specifically, these functions are realized by the following flow:
(1) A sample is injected into the HPLC-1 by the 233XL.
(2) The system is kept standing-by for a preset time (T1).
(3) The 233XL starts monitoring the signal from the UV detector.
(4) The system is kept standing-by until exceeding the preset UV level (peak detection).
(5) After the peak detection, the system is kept standing-by for a preset time (T2). Then fractionation inside the 233XL vial tubes is started.
(6) After a preset time (T3), the fractionation is finished.
(7) The obtained fraction is stirred by drawing and discharging.
(8) The fraction is partly injected into the HPLC-2.

The length of the column, which is filled with the anion-exchange resin, is not specifically limited. However, it is possible to shorten the column according to the particle diameter of the anion-exchange resin, the exchange capacity, or the like, so as to shorten the analysis time.

Abovementioned UV detector 14 equipped with the cell having a short optical path monitors the fraction eluted from the anion-exchange column (HPLC-1) 11, and detects the elution position of 8-OH-rGuo contained in the sample. By monitoring the elution position of 8-OH-rGuo by the UV detector 14, the elution time of 8-OH-dG can be obtained, along with which, by operating the column switching valve 16, the fraction containing 8-OH-dG can be reliably collected.

At the same time, the UV detector 14 equipped with a cell having a short optical path can measure 7-MG or Cre in the sample by adjusting the UV wavelength of the detector.

The above-mentioned reverse phase column (HPLC-2) 12 further purifies the fraction containing 8-OH-dG obtained from the anion-exchange column (HPLC-1), and the column is not specifically limited as long as it has the property of a reverse phase column. Examples of commercial products include YMC-Pack ODS-AM (S-5 μm) (manufactured by YMC Co., Ltd.), Shiseido Capcell Pac C18 MG (S-5 μm) (manufactured by Shiseido Co. Ltd.), and the like.

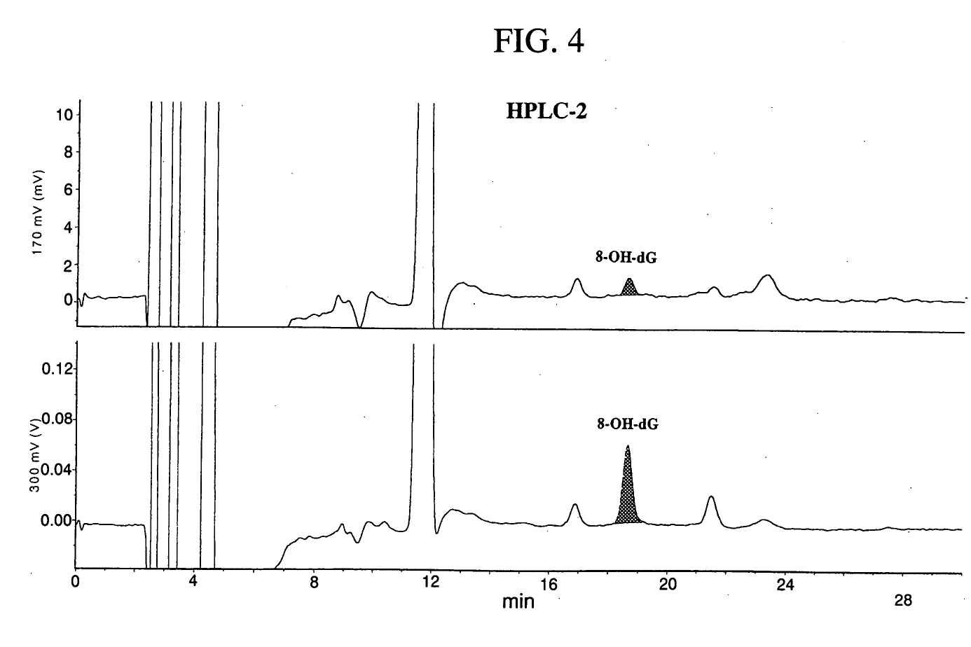

The above-mentioned detector 13 measures the purified 8-OH-dG obtained from the reverse phase column (HPLC-2), and is provided downstream of the reverse phase column (HPLC-2) 12. For the detector 13, an electrochemical detector (ECD), a liquid chromatography mass spectrometry (LCMS), and the like can be used. Concerning the electrochemical detector (ECD), the peak of 8-OH-dG appears in a characteristic ratio by selecting two kinds of preset voltages (FIG. 4), so that the peak can be identified to be 8-OH-dG.

Moreover, the analyzer for simultaneously analyzing 8-OH-dG and 7-MG or Cre relating to the embodiment of the present invention can process a large number of samples by continuous operation, in which case, the washing solution (solution C) of the guard column 35 is preferably a composition of 0.5M ammonium sulfate:acetonitrile=about 7:3.

As described above, according to the analyzer for simultaneously analyzing 8-OH-dG and 7-MG or Cre relating to the embodiment of the present invention, the anion-exchange column (HPLC-1) 11 specifically absorbs 8-OH-dG contained in the sample, and can remove almost all impurities contained in the sample at once.

Moreover, the analytical method to reliably fractionate the purified 8-OH-dG based on the elution position of a marker, such as 8-OH-rGuo, detected by the UV detector 14, is superior in terms of the recovery rate and reproducibility. The UV detector 14 having a short optical path can simultaneously measure 7-MG or Cre, which is a concentration correcting substance for 8-OH-dG, by adjusting the UV wavelength of the detector, as well. Consequently, the analyzer of the present embodiment enables the simultaneous analysis of 8-OH-dG and 7-MG or Cre.

Further, by continuous operation, a large amount of samples can be processed. Since the analyzer is relatively low in price, it is also superior in terms of economic efficiency.

(Analytical Method)

A description of the analytical method for 8-OH-dG and 7-MG or Cre using the analyzer shown in FIG. 1 follows.

(Determination of Fractionation Range (Time))

A mixture of 8-OH-dG, 8-OH-rGuo and urine was injected into the analyzer shown in FIG. 1 to pre-determine the fractionation range of 8-OH-dG.

By pre-determining the fractionation range, accurate elution time for 8-OH-dG can be obtained, along with which, by setting it to operate the column switching valve 16, the fraction containing 8-OH-dG can be reliably collected. As described above, in FIG. 1, if the sampling injector (231XL) is used instead of the automatic sampler 17, since the fractionation range (time) of 8-OH-dG is automatically determined by the peak detection based on the relative position relative to 8-OH-rGuo, it is not necessary to preset the fractionation range (time) of 8-OH-dG.

(Purification Method)

The purification method for 8-OH-dG relating to the present invention comprises a first purification step for purifying the sample by anion-exchange chromatography. As described above, a negatively charged oxidatively damaged guanine compound, such as 8-OH-rGuo or 8-OH-Gua, as well as 8-OH-dG can be easily purified and recovered by the anion-exchange chromatograph.

The elution conditions in the first purification step are preferably such that, the column temperature is from 50 to 65° C. and the flow rate is from 17 to 25 μl/min, in the case that the internal diameter of the column is 1 mm. For the simultaneous analysis of 8-OH-dG and 8-OH-Gua, as an eluent in the first purification step, an eluent containing carboxylic acid, such as formic acid, acetic acid or propionic acid (or salt thereof), can be cited. Among these, an eluent containing acetic acid is preferable.

In the purification method for 8-OH-dG of the present invention, it is possible to pre-add 8-OH-rGuo or the like to a sample as an internal standard marker for 8-OH-dG, so as to purify it. If 8-OH-rGuo is pre-added to the sample, after the elution of 8-OH-rGuo, 8-OH-dG is eluted after a fixed time, so by monitoring the elution position of 8-OH-rGuo by the UV detector 14, the accurate elution position (time) of 8-OH-dG can be obtained, and the fraction containing 8-OH-dG can be reliably collected.

Moreover, in the purification method for 8-OH-dG of the present invention, it is preferable to pre-add 8-OH-rGuo to the sample as an internal standard marker for 8-OH-dG so as to perform the first purification step by the anion-exchange chromatography, and to further purify the fraction containing 8-OH-dG obtained in the first purification step (second purification step).

For the second purification step, it is preferable to accomplish purification using reverse phase chromatography. Since the eluent (solution B) used for the reverse phase chromatography, the temperature condition, and the like vary depending on the reverse phase column (HPLC-2) 12 to be used, these are appropriately determined. In the case where human urine is analyzed using YMC-Pack ODS-AM (S-5 μm) (manufactured by YMC Co., Ltd.) as the reverse phase column, preferably, the column temperature is approximately 40° C., and the flow rate is approximately 0.9 ml/min.

(Measuring Method)

The measuring method for 8-OH-dG relating to the present invention comprises a measuring step for measuring the amount of the purified 8-OH-dG obtained by the purification method described above, wherein abovementioned electrochemical detector (ECD), and a liquid chromatography mass spectrometry (LCMS), and the like can be used for measuring the amount of the purified 8-OH-dG. Furthermore, the measuring method is applicable to measuring an oxidatively damaged guanine compound, such as 8-OH-rGuo (in the case of not adding as a marker), 8-OH-Gua, as well as 8-OH-dG.

In the case of continuous operation, both in the apparatus provided with the automatic sampler 17 as shown in FIG. 1 and in the apparatus provided with the sampling injector 27 as shown in FIG. 2, the elution position of 8-OH-dG is preferably checked regularly.

For the measurement of 7-MG or Cre relating to the present embodiment of the present invention, 7-MG or Cre in the injected sample is measured by the UV detector 14.

When the sample is urine from mice or rats, the wavelength of the UV detector for 7-MG measurement may be 254 nm. When the sample is human urine, in order to distinguish foreign substances from 7-MG, the wavelength of the UV detector may be longer than 254 nm, and preferably 300 to 310 nm. For the Cre measurement, the wavelength may be 235 to 260 nm, and preferably 245 nm, and a cell having a short optical path (preferably, approximately 0.2 mm) is used. The length of the optical path in the cell having a short optical path can be within the range of approximately 0.1 to 1 mm.

In the analytical method relating to the embodiment of the present invention, when urine is used as a sample, there are the following advantages:

In other words, urine is placed on a piece of filter paper and dried. Then, the urine is re-extracted from this piece of filter paper and analyzed.

Thus, urine can be collected at any location, and sending the dried pieces of filter paper to an analytical laboratory via postal mail enables efficient analysis of a large number of samples.

As described above, with the analytical method relating to the embodiment of the present invention, the adjustment of the UV wavelength of the UV detector 14 enables the measurement of 7-MG or Cre in a sample, enabling the simultaneous analysis of 8-OH-dG and 7-MG or Cre, which is a concentration correcting substance.

In the analytical method of the present invention, 8-OH-dG can be obtained with an excellent recovery rate. In addition, since the flow rate of the anion-exchange column (HPLC-1) in the first purification step is very low, the consumption of the eluent (solution A) and the washing solution (solution C) is extremely small, and the amount of the effluent waste after purification is also small, making the method preferable from the aspect of environmental protection. With the purification method for 8-OH-dG of the present invention, the purified 8-OH-dG can be reliably fractionated, and a fraction with almost no foreign substances can be obtained in the vicinity of the peak of 8-OH-dG. Moreover, if a method to detect the peak of a marker, such as 8-OH-rGuo, even with continuous operation, fractions containing 8-OH-dG can be reliably fractionated by corresponding to the shift of the fractionation rage per sample. Since the measuring method of the present invention measures the purified oxidatively damaged guanine compound, such as purified 8-OH-dG or 8-OH-Gua, obtained by the above purification method, it has high accuracy and reproducibility. Moreover, the continuous operation enables mass-processing.

Furthermore, in the analytical method of the present invention, 7-MG can be quantitatively measured, and 7-MG is generated in DNA due to a carcinogenic substance in cigarette smoke, and there is possibility that the present analytical method is applicable to a carcinogenesis risk evaluation method. In the analytical method of the present invention, when 7-MG is used for concentration correction, it is preferable to subtract the increase of 7-MG due to smoking in order to be precise.

Furthermore, the scope of the techniques of the present invention is not limited to the above embodiments. Various modifications can be made without departing from the spirit or scope of the present invention. For example, the composition of the eluents (solution A and solution B) and the washing solution (solution C), or the like may be appropriately modified corresponding to the columns (fillers) to be used.

The measuring method for an oxidatively damaged guanine compound of the present invention can be used in individual carcinogenesis risk evaluation, prediction and diagnosis of various disorders related to active oxygen (for example diabetes), evaluation of degree of aging or general health.

The evaluation method for the results obtained by the measuring method is described below using an example in the case of 8-OH-dG. As well as the urine sample, 8-OH-dG standard solution is periodically injected into the analyzer. These peak areas are then compared to calculate the 8-OH-dG concentration in the sample. The calculated 8-OH-dG concentration is then divided by the concentration of 7-MG or Cre simultaneously measured by the UV detector.

The analytical method of the present invention can be used in the clinical laboratories that analyze biological substance, such as urine. The analyzer of the present invention can be used in the field of analytical instrument manufacture.

### EXAMPLES

The following is a specific description of the present invention using examples. However the present invention is not considered as being limited to these.

(Preparation of Urine Sample)

One milliliter of human urine was placed in each of two Eppendorf tubes and frozen at −20° C. After the frozen urine was thawed and homogenized, 100 μl of each homogenate was diluted with the same volume of slightly acidic solution (composition; 96 ml of 0.6 mM sulfuric acid and 4 ml of acetonitrile), and 12 μg of 8-OH-rGuo were added. In addition, the pH was adjusted below 7 by adding 6.7 μl of 2M sodium acetate (pH 4.5). The mixture was well stirred and centrifuged at 15,000 rpm for 5 minutes, and the supernatant was made the urine sample. Furthermore, it is also possible to automate dilution, mixing and injection by directly placing the centrifuged urine on a sampling injector, such as 231XL.

(Purification and Fractionation of 8-OH-dG and Measurement of 7-MG or Cre)

Purification of 20 μl of the urine sample was accomplished by an anion-exchange column (MCI GEL CA08F, particle diameter 7 μm, sulfate type, internal diameter 1.5 mm, guard column length 4 cm, and main column length 12 cm). The column temperature was 65° C. and the flow rate was 50 μl/min., and 0.3 mM sulfuric acid (containing 2% acetonitrile) was used for an eluent.

According to this separation, 7-MG and Cre were detected using a UV detector (“UV/VIS-155” manufactured by Gilson) (absorption wavelength 305 nm and 245 nm), respectively. The peak recognition of 8-OH-rGuo in this analysis was conducted with 305 nm. The detection results are shown in FIG. 3.

As shown in FIG. 3, the peaks of 7-MG and Cre were detected, and it was clear that 7-MG and Cre could be measured according to the present example. Moreover, 8-OH-dG was eluted after having a fixed time difference from the 8-OH-rGuo elution. By monitoring the 8-OH-rGuo elution, the accurate elution position of 8-OH-dG could be ascertained so that the fraction containing 8-OH-dG could be reliably obtained.

(Purification and Measurement of 8-OH-dG)

The above fraction containing 8-OH-dG obtained by the anion-exchange chromatography was automatically injected into a reverse phase chromatography. Furthermore, Shiseido Capcell Pak C18 MG (S-5 μm) (250×4.6 mm) was used for the reverse phase column, and 10 mM of phosphate buffer (pH 6.7; the pH may slightly vary since it was prepared by diluting 0.1 M phosphate buffer (pH 6.7)) and 5% methanol (MeOH) eluent (solution B) were used. The column temperature was 40° C. and the flow rate was 1 ml/min. The separation pattern is shown in FIG. 4. Furthermore, the separation pattern was obtained using an electrochemical detector (“ESA Coulochem II” by ESA, Inc.) (voltage: 350 mV in guard cell; 170 mV at channel 1; 300 mV at channel 2).

As shown in FIG. 4, the peak of 8-OH-dG was detected, and it is clear that 8-OH-dG could be measured according to the present example. A value of (8-OH-dG/7-MG) or (8-OH-dG/Cre) is calculated from the peak area of 7-MG or Cre obtained from the above FIG. 3 and the peak area of 8-OH-dG obtained from FIG. 4, and this can be regarded as an index of the oxidative DNA damage.

(Simultaneous Analysis of 8-OH-dG and 8-OH-Gua)

Centrifuged human urine was placed to the sampling injector 231XL, and it was automatically diluted (1:1) with a slightly-acidic diluent (composition: 99 ml of 125 mM sodium acetate (pH 4.5) solution; 1 ml of acetonitrile) containing 8-OH-rGuo (120 μg/ml), mixed and injected into HPLC-1 (20 μl). For the column, an anion-exchange column (MCL GEL CA08F, particle diameter 7 μm, acetate type; internal diameter 1.5 mm, length of guard column 4 cm, and length of main column 12 cm) was used, and, the column temperature was 55° C., the flow rate was 50 μl/min, and 5 mM acetic acid containing 0.5% acetonitrile was used for an eluent. The 8-OH-dG and 8-OH-Gua fractions fractionated according to the peak recognition of 8-OH-rGuo were automatically injected into the HPLC-2. For the column, Innertsil ODS-3 (particle diameter 3 μm, internal diameter 4.6 mm, length 25 cm; manufactured by GL Sciences Inc.) was used, and, the column temperature was 30° C., the flow rate was 0.7 ml/min, and 10 mM sodium dihydrogenphosphate solution containing EDTA trisodium salt trihydrate (50 mg/l) and 8% methanol was used for an eluent. Furthermore, in this analytical example, ECD-300 manufactured by Eicom Corporation (Kyoto) was used for ECD (preset voltage: 550 mV).

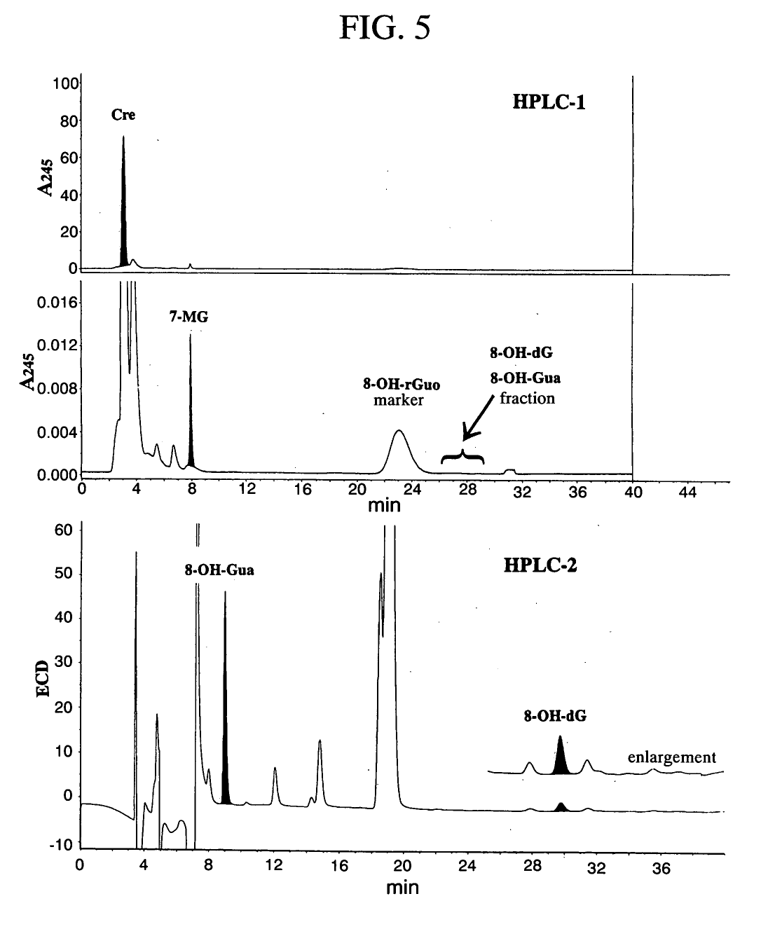

Results are shown in FIG. 5.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

1. An analytical method for an oxidatively damaged guanine compound comprising:
a step to purify an oxidatively damaged guanine compound generated as a result of damaging guanine in DNA, RNA or nucleotide using anion-exchange chromatography (HPLC-1); and
a step to measure the oxidatively damaged guanine compound by a detector.

2. The analytical method for an oxidatively damaged guanine compound according to claim 1, wherein the oxidatively damaged guanine compound is 8-hydroxydeoxyguanosine (8-OH-dG) and/or 8-hydroxyguanine (8-OH-Gua).

3. An analytical method for an oxidatively damaged guanine compound, wherein, the analytical method comprises:
a step to purify an oxidatively damaged guanine compound generated as a result of damaging guanine in DNA, RNA or nucleotide using anion-exchange chromatography (HPLC-1);
a step to measure a concentration correcting substance for the oxidatively damaged guanine compound contained in the sample using a detector; and
a step to measure the oxidatively damaged guanine compound by a detector, and
said oxidatively damaged guanine compound and the concentration correcting substance are simultaneously analyzed.

4. The analytical method for an oxidatively damaged guanine compound according to claim 3, wherein the oxidatively damaged guanine compound is 8-hydroxydeoxyguanosines (8-OH-dG) and/or 8-hydroxyguanine (8-OH-Gua), and the concentration correcting substance for the oxidatively damaged guanine compound is 7-methylguanine (7-MG) and/or creatinine (Cre).

5. An analytical method for an oxidatively damaged guanine compound, wherein, the analytical method comprises:
a step to purify an oxidatively damaged guanine compound generated as a result of damaging guanine in DNA, RNA or nucleotide using anion-exchange chromatography (HPLC-1);
a step to detect an elution position of a marker pre-added into the sample, and to appropriately measure the concentration correcting substance for the oxidatively damaged guanine compound contained in the sample; and
a step to measure the oxidatively damaged guanine compound by a detector, and the oxidatively damaged guanine compound and the concentration correcting substance are simultaneously analyzed.

6. The analytical method for an oxidatively damaged guanine compound according to claim 5, wherein the oxidatively damaged guanine compound is 8-hydroxydeoxyguanosine (8-OH-dG) and/or 8-hydroxyguanine (8-OH-Gua);
the concentration correcting substance for the oxidatively damaged guanine compound is 7-methylguanine (7-MG) and/or creatinine (Cre); and
the marker is 8-hydroxyguanosines (ribonucleosides) (8-OH-rGuo).

7. The analytical method for an oxidatively damaged guanine compound according to claim 1, wherein the sample is urine.

8. The analytical method for an oxidatively damaged guanine compound according to claim 3, wherein the sample is urine.

9. The analytical method for an oxidatively damaged guanine compound according to claim 5, wherein the sample is urine.

10. The analytical method for an oxidatively damaged guanine compound according to claim 7, wherein analysis is conducted by re-extracting the urine, which has been instilled onto a piece of paper and dried.

11. The analytical method for an oxidatively damaged guanine compound according to claim 8, wherein analysis is conducted by re-extracting the urine, which has been instilled onto a piece of paper and dried.

12. The analytical method for an oxidatively damaged guanine compound according to claim 9, wherein analysis is conducted by re-extracting the urine, which has been instilled onto a piece of paper and dried.

13. The analytical method for an oxidatively damaged guanine compound according to claim 1, 3 or 5, wherein the step to purify using the anion-exchange column (HPLC-1), a carboxylic acid type column and an eluent containing carboxylic acid or salt thereof are used.

14. The analytical method for an oxidatively damaged guanine compound according to claim 1, 3 or 5, further comprising:
a step to further purify a fraction containing the oxidatively damaged guanine compound purified by the anion-exchange column (HPLC-1) by a reverse phase column (HPLC-2); and
a step to measure the purified oxidatively damaged guanine compound purified by HPLC-2.

15. An analyzer for oxidatively damaged guanine compound provided with
1) an anion-exchange column (HPLC-1) that specifically absorbs an oxidatively damaged guanine compound generated as a result of damaging guanine in DNA, RNA or nucleotide contained in a sample;
2) a reverse phase column (HPLC-2) that further purifies a fraction containing the oxidatively damaged guanine compound obtained by purifying using the anion-exchange column (HPLC-1); and
3) a detector to be used for obtaining a fraction containing the oxidatively damaged guanine compound by the anion-exchange column (HPLC-1) and another detector that measures the purified oxidatively damaged guanine compound obtained by the reverse phase column (HPLC-2).

16. The analyzer according to claim 15 where the detector to be used for obtaining a fraction containing the oxidatively damaged guanine compound by the anion-exchange column (HPLC-1) is a detector equipped with a cell having a short optical path.

17. An analytical mechanism for an oxidatively damaged guanine compound (including a control program)
receiving a peak signal of a marker pre-added to a sample;
transmitting a signal to open a valve when the oxidative damaged guanine compound is eluted after a fixed time;
starting fractionation;
transmitting a fractionation completion signal after another fixed time; and
then transmitting a signal to inject the obtained fraction containing the oxidatively damaged guanine compound into a reverse phase column (HPLC-2), and
thereby purifying and recovering the oxidatively damaged guanine compound eluted from the reverse phase column (HPLC-2).
